(19) United States
(12) Patent Application Publication
(10) Pub. No.: US 2008/0243177 A1
(43) Pub. Date: Oct. 2, 2008

(54) Surgical instrument usable as a grasper and/or probe

(75) Inventors: Ran Oren, Kibbutz Gaaton (IL); Eran Zakai, Moshave Yodfat (IL); Wayne Poll, New Albany, OH (US); Dan Moor, Kibbutz Gaaton (IL)

Correspondence Address: Martin D. Moynihan;PRTSI, Inc., P.O. Box 16446, Arlington, VA, 22215, US

(73) Assignees: T.A.G. Medical Products, a Limited Partnership, Doar-Na Oshrat (IL); Ethicon Endo-Surgery Inc., Cincinnati, OH (US)

(21) Appl. No.: 11/729,919

(22) Filed: Mar. 30, 2007

(51) Int. Cl. A61B 17/50

(52) U.S. Cl. 606/210

(57) ABSTRACT

A finger-mountable surgical instrument usable as a tweezers or as a probe, includes a sleeve dimensioned to be received on the outer end of a finger of a user; an elongated arm having a proximal end fixed to the sleeve and a distal end projecting forwardly of the sleeve and defining a fixed jaw; a pivotal jaw pivotally mounted to the distal end of the elongated arm to open and closed positions; an actuator arm movably mounted with respect to the sleeve and coupled to the pivotal jaw such that moving the actuator arm in one direction moves the pivotal jaw to a closed position with respect to the fixed jaw, and moving the actuator arm in another direction moves the pivotal jaw to an open position with respect to the fixed jaw; and a locking arm carried by the sleeve and manually movable to a locking position for locking the pivotal jaw in its closed position with respect to the fixed jaw.

### FIELD AND BACKGROUND OF THE INVENTION

The present invention relates to a surgical instrument usable as a tweezers or as a probe. The invention is particularly useful in finger-mountable surgical instruments for use both in open surgery and in endoscopic surgery, and is therefore described below with respect to such instruments.

In an open surgery procedure, an incision is made through the skin, and the tissues overlying the surgical site are retracted in order to expose an area large enough to allow for access and orientation by direct vision. The surgeon may use a wide range of instruments to perform the specific procedure, and may also use the surgeon's fingers to touch organs and receive tactile feedback. However, fingers lack the delicacy to pick up fine tissue; they require making larger divisions if dissecting tissues; and they are subject to injury when holding tissue when sharp instruments or energy modalities, such as ultrasound or RF, are used to treat the surgical site.

In recent years, more and more procedures are being performed endoscopically. Endoscopy is a minimal invasive surgical procedure in which illumination, cameras and surgical tools are inserted into the patient's body via small incisions through the skin, and are manipulated externally, guided by the image displayed on a TV monitor. For the patient, trauma is minimized, and healing time and length of stay in hospital are shortened. Particularly, laparoscopy—endoscopy in the abdominal cavity—is being widely used both for diagnosis and for performing full surgical procedures.

However, several difficulties are inherent in this technique. Thus, considerable skill and experience are required to position the instruments spatially relative to internal organs while viewed in a two-dimensional monitor. Also, the field of vision displayable on the monitor is narrow making orientation even more difficult. In addition, it is hard to control the instruments because of their length, which is typically about 350 mm from the handles outside the body to the tip that actually performs the operations within the body cavity. This length is needed to reach the organs within the abdomen, when the cavity is insufflated as required by the technique. Moreover, direct contact between the organs and the surgeon's hand is not possible, so that tactile feedback is lost.

Recently, a modified laparoscopic technique has evolved which is referred to as Hand-Assisted Laparoscopic Surgery (HALS), in which one hand of the surgeon has access to the body cavity while maintaining insufflation. This technique, as described for example in U.S. Pat. No. 5,640,977 to Patrick Leahy et al, is now an alternative procedure of choice. As only a relatively small additional incision is required, just sufficient for admitting the surgeon's hand, the advantages of minimal invasiveness are preserved. For the surgeon, less training is required because the presence of his/her hand in the body cavity allows palpation of internal organs, biophysical feedback, and easier manipulation of various instruments within the body cavity while viewing the TV monitor.

With the advancement of the HALS technique, a need arose for instruments which could be mounted directly onto the fingers of the hand within the body cavity, i.e, the “ported” hand. Miniature forceps, graspers, scissors, dissectors, probes, retractors, etc., modeled on existing instruments used in open surgery and mounted on the surgeon's finger, could perform delicate surgical tasks and aid the laparoscopic instruments introduced through the “keyhole” incisions. Obviously it would be advantageous to provide such instruments for use in open procedures as well as in “keyhole” procedures.

Several of such instruments were suggested in U.S. Pat. No. 5,925,065 to Meyers et al, and US Application 20040193211 to Voegele et al. However, all the instruments described therein must be fixed to the finger outside of the body; therefore, when in HALS procedures the hand must be withdrawn through the port for changing instruments, which subsequently limits the advantages possible by this procedure.

### OBJECTS AND BRIEF SUMMARY OF THE PRESENT INVENTION

An object of the present invention is provide a surgical instrument of a construction enabling the instrument to be used as tweezers or as a probe during both open surgery and endoscopic surgery. Another object of the invention is to provide a finger-mountable surgical instrument which may be conveniently applied to a finger of the surgeon by using only the fingers of the surgeon's hand to receive the instrument, thereby making the instrument particularly useful in a HALS procedure since it eliminates the need for withdrawing the hand from the body cavity when applying the surgical instrument or when removing it.

According to one aspect of the present invention, there is provided a surgical instrument usable as a tweezers or as a probe, comprising: a surgical instrument usable as a tweezers or as a probe, comprising: an elongated arm having a proximal end and a distal end defining a fixed jaw; a pivotal jaw pivotally mounted to the distal end of the elongated arm to open and closed position with respect thereto; an actuator arm movably mounted with respect to the elongated arm coupled to the pivotal jaw such that moving the actuator arm in one direction moves the pivotal jaw to a closed position with respect to the fixed jaw, and moving the actuator arm in another direction moves the pivotal jaw to an open position with respect to the fixed jaw; and a locking arm carried by the elongated arm and manually movable to a locking position for locking the pivotal jaw in its closed position with respect to the fixed jaw. According to another aspect of the present invention, there is provided a finger-mountable surgical instrument usable as a tweezers or as a probe, comprising: a sleeve dimensioned to be received on the outer end of a finger of a user; an elongated arm having a proximal end fixed to the sleeve and a distal end projecting forwardly of the sleeve and defining a fixed jaw; a pivotal jaw pivotally mounted to the distal end of the elongated arm to open and closed position with respect thereto; an actuator arm movably mounted towards and away from the sleeve and coupled to the pivotal jaw such that moving the actuator arm towards the sleeve moves the pivotal jaw to a closed position with respect to the fixed jaw, and moving the actuator arm away from the sleeve moves the pivotal jaw to an open position with respect to the fixed jaw; a spring normally urging the actuator arm away from the sleeve, and thereby, the pivotal jaw to an open position with respect to the fixed jaw; and a locking arm carried by the sleeve and manually movable to a locking position for moving the pivotal jaw in its closed position with respect to the fixed jaw, and for locking the pivotal jaw in the closed position.

As will be described more particularly below, such a finger-mountable surgical instrument can serve both as tweezers and a probe, and can also be easily applied to a finger of the surgeon by using other fingers of the surgeon's same hand intended to carry the instrument. The novel construction enables the surgical instrument to be used in a HALS surgical procedure without removing the surgeon's hand from the body cavity when applying a surgical instrument to the surgeon's finger. Such a finger-mountable surgical instrument can also be produced in volume and at relatively low cost, thereby permitting one-time use if desired.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of finger-mountable surgical instrument constructed in accordance with the present invention, the illustrated instrument being shown in its open condition for use as a tweezers;

FIG. 2 is a side elevational view illustrating only the tweezers of FIG. 1;

FIG. 3 is an enlarged side elevational view illustrating the surgical instrument of FIG. 1; and

FIG. 4 is a view corresponding to that of FIG. 1 but illustrating the tweezers in its closed condition for use as a probe.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

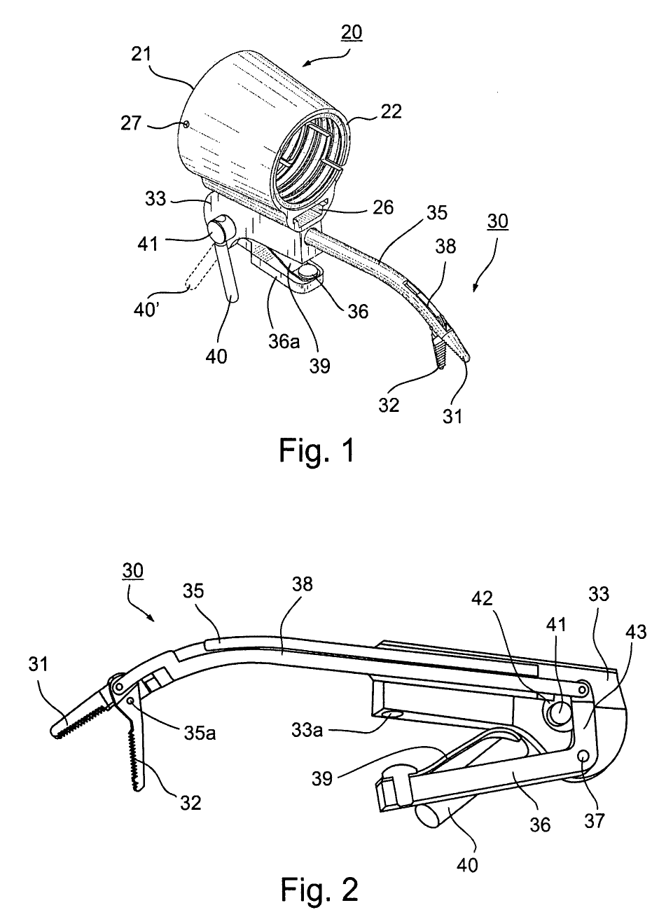

With reference to the drawings, there is illustrated a finger-mountable surgical instrument, including a sleeve, generally designated 20, dimensioned to be received on the outer end of a finger of a user (e.g., a surgeon), and surgical tweezers, generally designated 30, fixed to sleeve 20 and projecting forwardly of the sleeve for use by the surgeon in grasping tissue when the tweezers are in their open condition as illustrated in FIG. 1, or as a probe or retractor when the tweezers are in their closed condition in illustrated in FIG. 4. The illustrated instrument is constructed so that it may be quickly and conveniently applied to the index finger of one hand of the user by merely manipulating the instrument with other fingers of the same hand, thereby permitting the instrument to be used in a HALS operating procedure without removing the surgeon's hand from the body cavity.

Sleeve 20 has an open proximal end 21 (with the respect to the surgeon) for application to the outer end of the surgeon's finger, and an open distal end 22 which exposes the outer tip of the user's finger when mounted thereon. The surgical tweezers or probe 30 projects forwardly of distal end 22 of sleeve 20. Sleeve 20 is of a tapered construction, having an inner diameter and an outer diameter decreasing from its proximal end 21 to its distal end 22, which facilitates the application of the sleeve to the surgeon's finger.

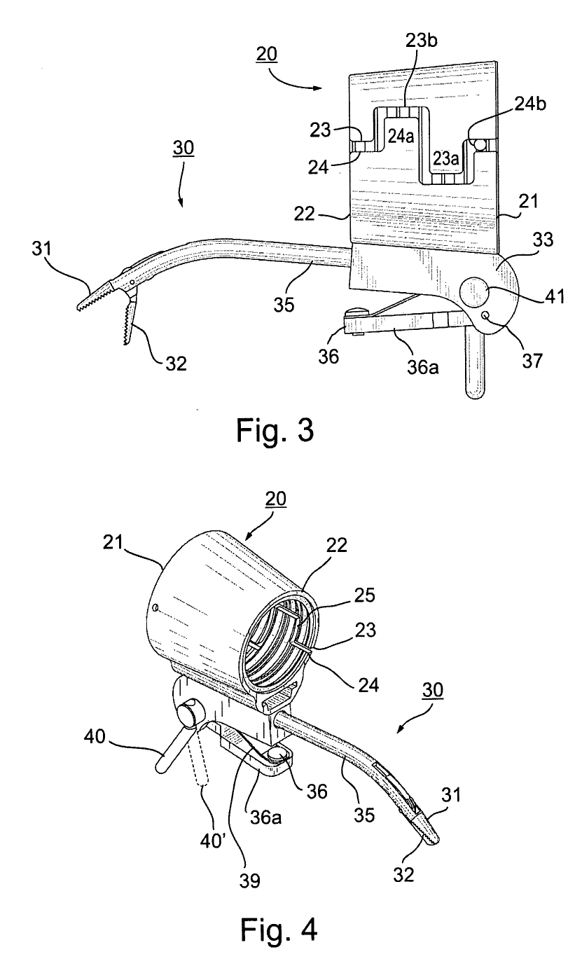

As shown particularly in FIG. 3, sleeve 20 is split along its longitudinal axis so as to define two opposed edges, 23, 24 confronting each other. The two edges 23, 24 include interengaging ribs (23a, 24a) and recesses (23b, 24b). Sleeve 20 is made of an elastic plastic material, such that the longitudinal-split permits its diameter to be increased in order to accommodate fingers of different thicknesses. The interengaging ribs and recesses not only better assure that the sleeve will firmly engage the entire outer surface of the surgeon's gloved finger when mounted thereon, but also facilitate the manipulation of the instrument onto the user's finger without pinching the finger.

As shown particularly in FIGS. 1 and 4, the inner surface of sleeve 20 is formed with circumferentially-extending ribs or rill-shaped undercuts 25 which better assure firm gripping of the surgeon's gloved finger when the sleeve is mounted thereon.

Sleeve 20 is further formed with a socket 26 for receiving another surgical instrument, if desired, e.g., a convention probe or retractor (not shown). A small hole 27 formed through sleeve 20, near its proximal end 21, serves for the attachment of a suture strand (not shown) which, when led through the port to the outside, facilitates the tracing of the instrument inside the body cavity during a HALS procedure.

As indicated earlier, the surgical instrument 30 illustrated in the drawings is a tweezers having a pair of jaws 31, 32, movable to an open position (FIG. 1) for grasping tissue and the like, or to a closed position (FIG. 4) to enable the tweezers to also serve as a surgical probe or retractor. The two jaws 31, 32 are carried by a body member 33 fixed to sleeve 20 in any suitable manner or integrally formed with the sleeve.

Body member 33 carries an elongated arm 35 having a proximal end fixed to the body member 33 (and thereby to sleeve 20), and a distal end projecting forwardly of the body member and defining fixed jaw 31. Elongated arm 35 is fixed to body member 33, e.g., via a screw 33a (FIG. 2). Jaw 32 is pivotally mounted to the distal end of elongated arm 35, about pivot point 35a, to an open position with respect to fixed jaw 31, as illustrated in FIGS. 1-3, or to a closed position as illustrated in FIG. 4.

An actuator arm 36 is pivotally mounted by a pin 37 towards and away from sleeve 20. The distal end of actuator arm 36 includes a finger piece 36a engageable by the surgeon's thumb, when the instrument is mounted on the surgeon's index finger, to depress the actuator arm towards sleeve 20. Actuator arm 36 is coupled by a rod 38 to pivotal jaw 32 such that pivoting actuator arm 36 towards sleeve 20 moves the pivotal jaw 32 to a closed position with respect to fixed jaw 31, and pivotting the actuator arm 36 away from the sleeve moves the pivotal jaw to an open position with respect to the fixed jaw 31.

Actuator arm 36 is normally urged by a leaf spring 39 away from sleeve 20, and thereby normally urges pivotal jaw 32 to its open position with respect to fixed jaw 31. However, depression of actuator arm 36 by the surgeon will move pivotal jaw 32 to its closed position, as illustrated in FIG. 2.

The illustrated surgical instrument further includes a locking arm 40 pivotally mounted, via pin 41, to body member 33 from a first position shown in full lines in FIG. 1, to a second position shown in full lines in FIG. 2. Locking lever 40 carries a cam 42 (FIG. 2) engageable with an extension 43 of actuator arm 36 to pivot the actuator arm to its jaw-closed condition, as illustrated in FIG. 4, when the locking arm is moved from its full-line position 40 illustrated in FIG. 1 to its full-line position 40 illustrated in FIG. 4.

The manner of using the surgical instrument illustrated in the drawings will be apparent from the above description.

Thus, the surgical instrument may be conveniently attached to the surgeon's index finger by merely inserting the gloved finger through the proximal end 21 of sleeve 20, until the sleeve firmly fits on the finger with the surgical instrument 30 projecting forwardly of the finger. The elasticity of the split sleeve 20 enables the sleeve to be conveniently applied to the surgeons' gloved finger and to accommodate fingers of different thicknesses, while the ribs 25 formed on the inner surface of the sleeve, firmly hold the surgical instrument on the surgeon's gloved finger.

Spring 39 normally urges actuator arm 36 to the position illustrated in FIG. 1, such that pivotal jaw 32 is in its open position with respect to fixed jaw 31. In this condition, locking arm 40 is in the full-line position illustrated in FIG. 1.

If it is desired to use the surgical instrument as tweezers for manipulating tissue, the surgeon, using his (or her) thumb, presses finger piece 36a of actuator arm 36 towards the sleeve 20, which thereby, via coupling rod 38, pivots jaw 32 to its closed position with respect to jaw 31, as illustrated in FIG. 2. Thus, whenever the surgeon wishes to grasp tissue, the surgeon would merely press actuator arm 36 towards sleeve 20, and whenever the surgeon wishes to release the tissue, the surgeon would merely release actuator arm 36, whereupon spring 39 urges the arm downwardly, and thereby urges jaw 32 to its open position.

If the surgeon desires to use the instrument as a probe or retractor, this may be done by pivoting locking arm 40, from its full-line position 40 illustrated in FIG. 1, to its full-line position 40 illustrated in FIG. 2. When locking arm is so pivoted, its cam 42 pivots actuator arm 36 to move the actuator arm towards sleeve 20, as if the actuator arm were being pressed by the surgeon, and thereby moves the pivotal jaw 32 to its closed position with respect to fixed jaw 31 and locks it in such position. As shown in the drawings, the outer tips of both jaws 31 and 32 are rounded, to thereby permit the two jaws, when in their closed positions, to better serve as a probe or retractor.

While the invention has been described with respect to one preferred embodiment, it will be appreciated that this is set forth merely for purposes of example, and that many other variations, modifications and applications of the invention may be made.

What is claimed is:

1. A surgical instrument usable as a tweezers or as a probe, comprising:
an elongated arm having a proximal end and a distal end defining a fixed jaw;
a pivotal jaw pivotally mounted to the distal end of said elongated arm to open and closed position with respect thereto;
an actuator arm movably mounted with respect to said elongated arm coupled to said pivotal jaw such that moving said actuator arm in one direction moves said pivotal jaw to a closed position with respect to said fixed jaw, and moving said actuator arm in another direction moves said pivotal jaw to an open position with respect to said fixed jaw;
and a locking arm carried by said elongated arm and manually movable to a locking position for locking said pivotal jaw in its closed position with respect to said fixed jaw.

2. The surgical instrument according to claim 1, wherein said actuator arm is pivotable towards said elongated arm to pivot the pivotal jaw to its closed position, and said locking arm is pivotable away from the said elongated arm to lock the pivotal jaw in its closed position.

3. The surgical instrument according to claim 1, wherein said instrument further includes a body member fixed to said proximal end of the elongated arm.

4. The surgical instrument according to claim 3, wherein said actuator arm and said locking arm are pivotally mounted about parallel axes on said body member.

5. The surgical instrument according to claim 3, wherein said actuator arm is coupled to said pivotal jaw via a coupling element extending along said elongated arm and connecting an end of said actuator arm to said pivotal jaw.

6. The surgical instrument according to claim 3, wherein an end of said actuator arm includes a fingerpiece engageable by the user's thumb to depress same towards said body member.

7. The surgical instrument according to claim 3, wherein said locking arm includes a cam element effective, when moved to its locking position, to move said actuator arm to, and to lock it in, the jaw-closed position.

8. The surgical instrument according to claim 1, wherein the distal end of said elongated arm defining said fixed jaw is curved away from its longitudinal axis.

9. The surgical instrument according to claim 1, wherein the distal tip of said fixed and pivotal jaws are rounded to facilitate using the instrument as a probe when the jaws are in their closed positions.

10. The surgical instrument according to claim 1, further comprising a spring normally urging said actuator arm to move said pivotal jaw to an open position with respect to said fixed jaw.

11. The surgical instrument according to claim 1, wherein the proximal end of said elongated arm is fixed to a sleeve dimensioned to be received on the outer end of the finger of a user.

12. A finger-mountable surgical instrument usable as a tweezers or as a probe, comprising:
a sleeve dimensioned to be received on the outer end of a finger of a user;
an elongated arm having a proximal end fixed to said sleeve and a distal end projecting forwardly of said sleeve and defining a fixed jaw;
a pivotal jaw pivotally mounted to the distal end of said elongated arm to open and closed position with respect thereto;
an actuator arm movably mounted towards and away from said sleeve and coupled to said pivotal jaw such that moving said actuator arm towards said sleeve moves said pivotal jaw to a closed position with respect to said fixed jaw, and moving said actuator arm away from said sleeve moves said pivotal jaw to an open position with respect to said fixed jaw;
a spring normally urging said actuator arm away from said sleeve and, thereby, said pivotal jaw to an open position with respect to said fixed jaw;
and a locking arm carried by said sleeve and manually movable to a locking position for moving said pivotal jaw to its closed position with respect to said fixed jaw, and for locking the pivotal jaw in said closed position.

13. The surgical instrument according to claim 12, wherein said actuator arm is pivotable towards its sleeve to pivot the pivotal jaw to its closed position, and said locking arm is pivotable away from the said sleeve to move the pivotal jaw to its closed position and for locking it in said closed position.

14. The surgical instrument according to claim 12, wherein said instrument further includes a body member fixed to said sleeve; said proximal end of the elongated arm being fixed to said body member and projecting forwardly thereof to define said fixed jaw.

15. The surgical instrument according to claim 14, wherein said actuator arm and said locking arm are pivotally mounted about said parallel axes on said body member.

16. The surgical instrument according to claim 14, wherein said actuator arm is coupled to said pivotal jaw via a coupling element extending along said elongated arm and connecting an end of said actuator arm to said pivotal jaw.

17. The surgical instrument according to claim 14, wherein an end of said actuator arm includes a fingerpiece engageable by the user's thumb, when the instrument is mounted on the user's finger, to depress the fingerpiece towards said sleeve.

18. The surgical instrument according to claim 14, wherein said locking arm includes a cam element effective, when moved to its locking position, to move said actuator arm and to lock it in the jaw-closed position.

19. The surgical instrument according to claim 11, wherein the distal end of said elongated arm defining said fixed jaw and mounting the pivotal jaw, is curved away from its longitudinal axis.

20. The surgical instrument according to claim 11, wherein the distal tip of said fixed and pivotal jaws are rounded to facilitate using the instrument as a probe when the jaws are in their closed positions.
